SEQUENCE LISTING (30 sequences)

SEQ ID NO: 1 - DNA, 27 bp, Homo sapiens
  misc_feature: artificial DNA
tgtcctctgtgatattcctctgagttg

SEQ ID NO: 2 - DNA, 26 bp, Homo sapiens
  misc_feature: artificial DNA
gtggatgttacacaggaggaagagag

SEQ ID NO: 3 - DNA, 24 bp, Homo sapiens
  misc_feature: artificial DNA
ccctctatttagtcatttgaaaac

SEQ ID NO: 4 - DNA, 22 bp, Homo sapiens
  misc_feature: artificial DNA
gaatggtatcctcataatactc

SEQ ID NO: 5 - DNA, 23 bp, Homo sapiens
  misc_feature: artificial DNA
ctccacacccaggtccacacatt

SEQ ID NO: 6 - DNA, 24 bp, Homo sapiens
  misc_feature: artificial DNA
aggtctctgtagtcaaatcctata

SEQ ID NO: 7 - DNA, 22 bp, Homo sapiens
cctggaaccacagtatttaagg

SEQ ID NO: 8 - DNA, 22 bp, Homo sapiens
ccttaaatactgtggttccagg

SEQ ID NO: 9 - DNA, 21 bp, Homo sapiens
tgcttttcatgaggaacaagg

SEQ ID NO: 10 - DNA, 21 bp, Homo sapiens
ccttgttcctcatgaaaagca

SEQ ID NO: 11 - DNA, 21 bp, Homo sapiens
tcctctttgcagaaaagcggt

SEQ ID NO: 12 - DNA, 21 bp, Homo sapiens
accgcttttctgcaaagagga

SEQ ID NO: 13 - DNA, 21 bp, Homo sapiens
tcattgtactattgcagtatt

SEQ ID NO: 14 - DNA, 21 bp, Homo sapiens
aatactgcaatagtacaatga

SEQ ID NO: 15 - DNA, 21 bp, Homo sapiens
ccaggtgatcacaaatggtaa

SEQ ID NO: 16 - DNA, 21 bp, Homo sapiens
ttaccatttgtgatcacctgg

SEQ ID NO: 17 - DNA, 21 bp, Homo sapiens
tctttttgaagagttatatct

SEQ ID NO: 18 - DNA, 21 bp, Homo sapiens
agatataactcttcaaaaaga

SEQ ID NO: 19 - protein, 10 aa, Homo sapiens
KTAFHEEQGH

SEQ ID NO: 20 - protein, 11 aa, Homo sapiens
FFPLCRKAVEM

SEQ ID NO: 21 - protein, 11 aa, Homo sapiens
EIPGTTVFKSS

SEQ ID NO: 22 - protein, 11 aa, Homo sapiens
NISLYYCSIFD

SEQ ID NO: 23 - protein, 11 aa, Homo sapiens
INPGDHKCYAD

SEQ ID NO: 24 - protein, 11 aa, Homo sapiens
DCLFEELYLLT

SEQ ID NO: 25 - DNA, 32 bp, Homo sapiens
  misc_feature: artificial DNA
actgctaagaataataggttttcatttagttc

SEQ ID NO: 26 - DNA, 26 bp, Homo sapiens
  misc_feature: artificial DNA
gccacagatgcactgtgactcgggag

SEQ ID NO: 27 - DNA, 25 bp, Homo sapiens
  misc_feature: artificial DNA
taccactgactgggtgtgtgtctga

SEQ ID NO: 28 - DNA, 28 bp, Homo sapiens
  misc_feature: artificial DNA
ctcaatccagaaagacttcatacctgtt

SEQ ID NO: 29 - DNA, 29 bp, Homo sapiens
  misc_feature: artificial DNA
aatccctgatgtcattcttcatagtattt

SEQ ID NO: 30 - DNA, 31 bp, Homo sapiens
  misc_feature: artificial DNA
catccattacattttcaggctttagcataat

(19) United States
(12) Patent Application Publication
(10) Pub. No.: US 2006/0078879 A1
(43) Pub. Date: Apr. 13, 2006

(54) Polymorphisms in the human gene for tpmt and their use in diagnostic and therapeutic applications

(75) Inventors: Matthias Schwab, Stuttgart (DE); Elke Schaeffeler, Stuttgart (DE)

Correspondence Address: FOLEY AND LARDNER LLP;SUITE 500, 3000 K STREET NW, WASHINGTON, DC, 20007, US

(73) Assignee: Epidauros Biotechnologie AG

(21) Appl. No.: 10/503,078

(22) PCT Filed: Feb. 4, 2003
(86) PCT No.: PCT/EP03/01090

§ 371 Date: Jun. 13, 2005

(30) Foreign Application Priority Data
Feb. 4, 2002 (EP) 02001978.2

(51) Int. Cl. C12Q 1/68; C07H 21/04; C12P 21/06; C12N 9/10; C07K 16/40

(52) U.S. Cl. 435/6; 435/69.1; 435/320.1; 435/325; 435/193; 536/23.2; 530/388.26

(57) ABSTRACT

The present invention relates to a polymorphic TPMT polynucleotide. Moreover, the invention relates to genes or vectors comprising the polynucleotides of the invention and to a host cell genetically engineered with the polynucleotide or gene of the invention. Further, the invention relates to methods for producing molecular variant polypeptides or fragments thereof, methods for producing cells capable of expressing a molecular variant polypeptide and to a polypeptide or fragment thereof encoded by the polynucleotide or the gene of the invention or which is obtainable by the method or from the cells produced by the method of the invention. Furthermore, the invention relates to an antibody which binds specifically the polypeptide of the invention. Moreover, the invention relates to a transgenic non-human animal. The invention also relates to a solid support comprising one or a plurality of the above mentioned polynucleotides, genes, vectors, polypeptides, antibodies or host cells. Furthermore, methods of identifying a polymorphism, identifying and obtaining a pro-drug or drug or an inhibitor are also encompassed by the present invention. In addition, the invention relates to methods for producing of a pharmaceutical composition and to methods of diagnosing a disease. Further, the invention relates to a method of detection of the polynucleotide of the invention. Furthermore, comprised by the present invention are a diagnostic and a pharmaceutical composition. Even more, the invention relates to uses of the polynucleotides, genes, vectors, polypeptides or antibodies of the invention for the preparation of pharmaceutical or diagnostic compositions. Finally, the invention relates to a diagnostic kit.

The present invention relates to a polymorphic TPMT polynucleotide. Moreover, the invention relates to genes or vectors comprising the polynucleotides of the invention and to a host cell genetically engineered with the polynucleotide or gene of the invention. Further, the invention relates to methods for producing molecular variant polypeptides or fragments thereof, methods for producing cells capable of expressing a molecular variant polypeptide and to a polypeptide or fragment thereof encoded by the polynucleotide or the gene of the invention or which is obtainable by the method or from the cells produced by the method of the invention. Furthermore, the invention relates to an antibody which binds specifically the polypeptide of the invention. Moreover, the invention relates to a transgenic non-human animal. The invention also relates to a solid support comprising one or a plurality of the above mentioned polynucleotides, genes, vectors, polypeptides, antibodies or host cells. Furthermore, methods of identifying a polymorphism, identifying and obtaining a pro-drug or drug or an inhibitor are also encompassed by the present invention. In addition, the invention relates to methods for producing of a pharmaceutical composition and to methods of diagnosing a disease. Further, the invention relates to a method of detection of the polynucleotide of the invention. Furthermore, comprised by the present invention are a diagnostic and a pharmaceutical composition. Even more, the invention relates to uses of the polynucleotides, genes, vectors, polypeptides or antibodies of the invention for the preparation of pharmaceutical or diagnostic compositions. Finally, the invention relates to a diagnostic kit.

Thiopurine S-methyltransferase (TPMT) is a cytosolic enzyme which catalyzes the S-methylation of aromatic and heterocyclic sulfhydryl compounds such as 6-mercaptopurine, 6-thioguanine and azathioprine, collectively termed as thiopurines (Krynetski, Pharm Res 16 (1999), 342-9; Lennard, Br J Clin Pharmacol 47 (1999), 131-43.) These drugs are used in the treatment of acute lymphoblastic leukaemia (ALL), autoimmune disorders, inflammatory bowel disease and organ transplant recipients. The metabolism of these drugs is severely affected by the genetic polymorphism of TPMT (Krynetski, Pharm Res 16 (1999), 342-9; Evans, J Clin Oncol 19 (2001), 2293-301; Hon, Hum Mol Genet 8 (1999), 371-6; Krynetski, Mol Pharmacol 47 (1995), 1141-7; Krynetski, Proc Natl Acad Sci USA 92 (1995), 949-53; Krynetski, Pharmacogenetics 6 (1996), 279-90; Krynetski, Pharmacology 61 (2000), 136-46; Otterness, Clin Pharmacol Ther 62 (1997), 60-73; Kubota, Br J Clin Pharmacol 51 (2001), 475-7; Loennechen, Clin Pharmacol Ther 70 (2001), 183-8; Rossi, Eur J Clin Pharmacol 57 (2001), 51-4; Spire-Vayron de la Moureyre, Hum Mutat 12 (1998), 177-85; Spire-Vayron de la Moureyre, Br J Pharmacol 125 (1998), 879-87; Hiratsuka, Biol Pharm Bull 23 (2000), 1131-5; Hiratsuka, Biol Pharm Bull 23 (2000), 1090-3; Hiratsuka, Mutat Res 448 (2000), 91-5; Collie-Duguid, Pharmacogenetics 9 (1999), 37-42; Ameyaw, Hum Mol Genet 8 (1999), 367-70; Kumagai, Pharmacogenetics 11 (2001), 275-8; McLeod, Pharmacogenetics 9 (1999), 773-6; Tai, Am J Hum Genet 58 (1996), 694-702; Tai, Proc Natl Acad Sci USA 94 (1997), 6444-9). Several case reports and clinical studies have shown that patients with exceptionally low TPMT activity (approximately 1 in 300 individuals) are at high risk of developing severe and potentially fatal hematopoietic toxicity (e.g., pancytopenia), caused by the accumulation of cytotoxic metabolites after treatment with standard doses of thiopurines, while subjects with very high activity may be undertreated (Evans, J Clin Oncol 19 (2001), 2293-301; Krynetski, Pharmacogenetics 6 (1996), 279-90; Lennard, Lancet 336 (1990), 225-9; Leipold, Arthritis Rheum 40 (1997), 1896-8; Schutz, Lancet 341 (1993), 436; Weinshilboum, Annu Rev Pharmacol Toxicol 39 (1999), 19-52). Additionally, recent data indicate that patients with heterozygous genotypes, constituting about 10% of Caucasian and African-American populations, are also at greater risk of thiopurine toxicity (Evans, J Clin Oncol 19 (2001), 2293-301; Relling, J Natl Cancer Inst 91 (1999), 2001-8). Prospective determination of erythrocyte TPMT activity is therefore emerging as a routine safety measure prior to therapy to avoid drug toxicity, but there are a number of different limitations with respect to the determination of the constitutive TPMT enzyme activity. For example, if a deficient or heterozygous patient has received transfusions with red blood cells (RBC) from a homozygous wild-type individual (a rather likely case), TPMT activity cannot be reliably determined within 30-60 days after transfusion (Krynetski, Pharmacology 61 (2000), 136-46; Schwab, Gastroenterology 121 (2001), 498-9). Furthermore, thiopurine administration itself may alter TPMT activity in erythrocytes with an increase of enzyme activity (Lowry, Gut 49 (2001), 656-64). Some other clinically important drugs (e.g., sulfasalazine, olsalazine) are partly potent inhibitors of TPMT, resulting in a possible misclassification especially for heterozygous patients (Lennard, Br J Clin Pharmacol 47 (1999), 131-43).

Several mutant alleles responsible for TPMT deficiency have been described and the relationship between TPMT geno- and phenotype has been most clearly defined for the clinically relevant TPMT alleles *2, *3A and *3C in patients and healthy subjects (Evans, J Clin Oncol 19 (2001), 2293-301). Although ten mutant alleles are known to be associated with intermediate or low activity, molecular diagnosis by genotyping can not predict the TPMT phenotype in 100%. Only 85-95% concordance of genotype and phenotype can be achieved (McLeod, Leukemia 14 (2000), 567-572; Yates, Ann Intern Med 126 (1997), 608-614).

Thus, means and methods for reliably diagnosing and treating diseases, drug response and disorders based on dysfunctions or dysregulations of TPMT were not available yet but are nevertheless highly desirable. Thus, the technical problem underlying the present invention is to comply with the above specified needs.

The solution to this technical problem is achieved by providing the embodiments characterized in the claims.

Accordingly, the present invention relates to a polynucleotide comprising a polynucleotide selected from the group consisting of:
(a) a polynucleotide having the nucleic acid sequence of SEQ ID NO: 7 to 18;
(b) a polynucleotide encoding a polypeptide having the amino acid sequence of SEQ ID NO: 19 to 24;
(c) a polynucleotide capable of hybridizing to a TPMT gene, wherein said polynucleotide is having a substitution at a position corresponding to position 488 of the TPMT gene (GenBank Accession No: AF019364.1); or at a position corresponding to position 516 of the TPMT gene (GenBank Accession No: AF019367.1); or at a position corresponding to position 391 of the TPMT gene (GenBank Accession No: AF019365.1); or at a position corresponding to position 463 of the TPMT gene (GenBank Accession No: AF019366.1), or at a position corresponding to position 1236 of the TPMT gene (GenBank Accession No: AF019367.1); or at a position corresponding to position 679 of the TPMT gene (GenBank Accession No: AF019369.1);
(d) a polynucleotide capable of hybridizing to a TPMT gene, wherein said polynucleotide is having a G at a position corresponding to position 488 of the TPMT gene (GenBank Accession No: AF019364.1) or an A at a position corresponding to position 391 of the TPMT gene (GenBank Accession No: AF019365.1) or an A at a position corresponding to position 516 or 1236 of the TPMT gene (GenBank Accession No: AF019367.1) or a C at a position corresponding to position 463 of the TPMT gene (GenBank Accession No: AF019366.1) or a G at a position corresponding to position 679 of the TPMT gene (GenBank Accession No: AF019369.1);
(e) a polynucleotide encoding an TPMT polypeptide or fragment thereof, wherein said polypeptide comprises an amino acid substitution at position 42, 71, 119, 132, 163 or 238 of the TPMT polypeptide (GenBank Accession No: AAC51865.1);
(f) a polynucleotide encoding an TPMT polypeptide or fragment thereof, wherein said polypeptide comprises an amino acid substitution of Q to E at position 42 of the TPMT polypeptide (GenBank Accession No: AAC51865.1) or G to R at position 71, or K to T at position 119 or C to Y at position 132 or R to H at position 163 or K to E at position 238 of the TPMT polypeptide (GenBank Accession No: AAC51865.1).

In the context of the present invention the term “polynucleotides” or the term “polypeptides” refers to different variants of a polynucleotide or polypeptide. Said variants comprise a reference or wild type sequence of the polynucleotides or polypeptides of the invention as well as variants which differ therefrom in structure or composition. In the following, said variants are sometimes also referred to as variant alleles. Reference or wild type sequences for the polynucleotides are GenBank Accession Nos: AF019364.1 AF019365.1, AF019367.1, and AF019369.1.

Reference or wild type sequence for the polypeptides of the invention is GenBank Accession No: AAC51865.1. The differences in structure or composition usually occur by way of nucleotide or amino acid substitution(s).

Preferably, said nucleotide substitution(s) comprised by the present invention result(s) in one or more changes of the corresponding amino acid(s) of the polypeptides of the invention. The variant polynucleotides and polypeptides also comprise fragments of said polynucleotides or polypeptides of the invention. The polynucleotides and polypeptides as well as the aforementioned fragments thereof of the present invention are characterized as being associated with a TPMT dysfunction or dysregulation comprising, e.g., insufficient and/or altered metabolism. Said dysfunctions or dysregulations referred to in the present invention cause a disease or disorder or a prevalence for said disease or disorder. Preferably, as will be discussed below in detail, said disease is thiopurine-induced toxicity, such as myelosupression, e.g. pancytopenia, leucopenia, thrombocytopenia, anemia or any other disease caused by a dysfunction or dysregulation due to a polynucleotide or polypeptides of the invention, also referred to as TPMT gene associated diseases in the following.

The term “hybridizing” as used herein refers to polynucleotides which are capable of hybridizing to the polynucleotides of the invention or parts thereof which are associated with a TPMT dysfunction or dysregulation. Thus, said hybridizing polynucleotides are also associated with said dysfunctions and dysregulations. Preferably, said polynucleotides capable of hybridizing to the polynucleotides of the invention or parts thereof which are associated with TPMT dysfunctions or dysregulations are at least 70%, at least 80%, at least 95% or at least 100% identical to the polynucleotides of the invention or parts thereof which are associated with TPMT dysfunctions or dysregulations. Therefore, said polynucleotides may be useful as probes in Northern or Southern Blot analysis of RNA or DNA preparations, respectively, or can be used as oligonucleotide primers in PCR analysis dependent on their respective size. Also comprised by the invention are hybridizing polynucleotides which are useful for analysing DNA-Protein interactions via, e.g., electrophoretic mobility shift analysis (EMSA). Preferably, said hybridizing polynucleotides comprise at least 10, more preferably at least 15 nucleotides in length while a hybridizing polynucleotide of the present invention to be used as a probe preferably comprises at least 100, more preferably at least 200, or most preferably at least 500 nucleotides in length.

It is well known in the art how to perform hybridization experiments with nucleic acid molecules, i.e. the person skilled in the art knows what hybridization conditions she has to use in accordance with the present invention. Such hybridization conditions are referred to in standard text books such as Molecular Cloning A Laboratory Manual, Cold Spring Harbor Laboratory (1989) N.Y. Preferred in accordance with the present inventions are polynucleotides which are capable of hybridizing to the polynucleotides of the invention or parts thereof which are associated with a TPMT dysfunction or dysregulation under stringent hybridization conditions, i.e. which do not cross hybridize to unrelated polynucleotides such as polynucleotides encoding a polypeptide different from the TPMT polypeptides of the invention. Preferably, stringent hybridization conditions refer to an overnight incubation at 42° C. in 50% Formamid/10×SSC followed by three washing steps in 1×SSC/0.2% SDS, 0.1×SSC/0.2% SDS and 0.1×SSC.

The term “corresponding” as used herein means that a position is not only determined by the number of the preceding nucleotides and amino acids, respectively. The position of a given nucleotide or amino acid in accordance with the present invention which may be substituted may vary due to deletions or additional nucleotides or amino acids elsewhere in the gene or the polypeptide. Thus, under a “corresponding position” in accordance with the present invention it is to be understood that nucleotides or amino acids may differ in the indicated number but may still have similar neighboring nucleotides or amino acids. Said nucleotides or amino acids which may be exchanged, deleted or comprise additional nucleotides or amino acids are also comprised by the term “corresponding position”. Said nucleotides or amino acids may for instance together with their neighbors form sequences which may be involved in the regulation of gene expression, stability of the corresponding RNA or RNA editing, as well as encode functional domains or motifs of the protein of the invention.

In accordance with the present invention, the mode and population distribution of genetic variations in the TPMT gene has been analyzed by sequence analysis of relevant regions of the human said gene from many different individuals. It is a well known fact that genomic DNA of individuals, which harbor the individual genetic makeup of all genes, including the TPMT gene, can easily be purified from individual blood samples. These individual DNA samples are then used for the analysis of the sequence composition of the alleles of the TPMT gene that are present in the individual which provided the blood sample. The sequence analysis was carried out by PCR amplification of relevant regions of said genes, subsequent purification of the PCR products, followed by automated DNA sequencing with established methods (e.g. ABI dyeterminator cycle sequencing).

One important parameter that had to be considered in the attempt to determine the individual genotypes and identify novel variants of the TPMT gene by direct DNA-sequencing of PCR-products from human blood genomic DNA is the fact that each human harbors (usually, with very few abnormal exceptions) two gene copies of each autosomal gene (diploidy). Because of that, great care had to be taken in the evaluation of the sequences to be able to identify unambiguously not only homozygous sequence variations but also heterozygous variations. The details of the different steps in the identification and characterization of novel polymorphisms in the TPMT gene (homozygous and heterozygous) are described in the Examples below.

Over the past 20 years, genetic heterogeneity has been increasingly recognized as a significant source of variation in drug response. Many scientific communications (Meyer, Ann. Rev. Pharmacol. Toxicol. 37 (1997), 269-296 and West, J. Clin. Pharmacol. 37 (1997), 635-648) have clearly shown that some drugs work better or may even be highly toxic in some patients than in others and that these variations in patient's responses to drugs can be related to molecular basis. This “pharmacogenomic” concept spots correlations between responses to drugs and genetic profiles of patient's (Marshall, Nature Biotechnology, 15 (1997), 954-957; Marshall, Nature Biotechnology, 15 (1997), 1249-1252). In this context of population variability with regard to drug therapy, pharmacogenomics has been proposed as a tool useful in the identification and selection of patients which can respond to a particular drug without side effects. This identification/selection can be based upon molecular diagnosis of genetic polymorphisms by genotyping DNA from leukocytes in the blood of patient, for example, and characterization of disease (Bertz, Clin. Pharmacokinet. 32 (1997), 210-256; Engel, J. Chromatogra. B. Biomed. Appl. 678 (1996), 93-103). For the founders of health care, such as health maintenance organizations in the US and government public health services in many European countries, this pharmacogenomics approach can represent a way of both improving health care and reducing overheads because there is a large cost to unnecessary drugs, ineffective drugs and drugs with side effects.

The mutations in the variant genes of the invention sometime result in amino acid substitution(s) either alone or in combination. It is, of course, also possible to genetically engineer such mutations in wild type genes or other mutant forms. Methods for introducing such modifications in the DNA sequence of said genes are well known to the person skilled in the art; see, e.g., Sambrook, Molecular Cloning A Laboratory Manual, Cold Spring Harbor Laboratory (1989) N.Y.

For the investigation of the nature of the alterations in the amino acid sequence of the polypeptides of the invention may be used such as BRASMOL that are obtainable from the Internet. Furthermore, folding simulations and computer redesign of structural motifs can be performed using other appropriate computer programs (Olszewski, Proteins 25 (1996), 286-299; Hoffman, Comput. Appl. Biosci. 11 (1995), 675-679). Computers can be used for the conformational and energetic analysis of detailed protein models (Monge, J. Mol. Biol. 247 (1995), 995-1012; Renouf, Adv. Exp. Med. Biol. 376 (1995), 37-45). These analysis can be used for the identification of the influence of a particular mutation on S-methylation of drugs.

Usually, said amino acid substitution in the amino acid sequence of the protein encoded by the polynucleotide of the invention is due to one or more nucleotide substitution(s) or any combinations thereof. Preferably said nucleotide substitutions, may result in an amino acid substitution of Q to E at position 42 of the TPMT polypeptide (GenBank Accession No: AAC51865.1) or in an amino acid substitution G to R at position 71 of the TPMT polypeptide or in an amino acid substitution of K to T at position 119 of the TPMT polypeptide or in an amino acid substitution of C to Y at position 132 of the TPMT polypeptide or in an amino acid substitution of R to H at position 163 of the TPMT polypeptide or in an amino acid substitution K to E at position 238 of the TPMT polypeptide (GenBank Accession No: AAC51865.1). The polypeptides of encoded by the polynucleotides of the invention have altered biological or immunological properties due to the mutations referred to in accordance with the present invention. Examples for said altered properties are stability of the polypeptides which may be effected or an altered enzyme activity or substrate specificity characterized by altered drug metabolism.

The mutations in the TPMT gene detected in accordance with the present invention are listed in Table 2. The methods of the mutation analysis followed standard protocols and are described in detail in the Examples. In general, such methods are to be used in accordance with the present invention for evaluating the phenotypic spectrum as well as the overlapping clinical characteristics of diseases or conditions related to dysfunctions or dysregulations and diseases related to impaired enzyme activity. Advantageously, the characterization of said mutants may form the basis of the development of molecular diagnosis assays, which can predict the TPMT phenotype. Patients can be screened for the presence of the above mentioned mutants before starting drug therapy, preventing them from developing severe toxicities.

Said methods encompass for example haplotype analysis, single-strand conformation polymorphism analysis (SSCA), PCR and direct sequencing. On the basis of thorough clinical characterization of many patients the phenotypes can then be correlated to these mutations.

Also comprised by the polynucleotides referred to in the present invention are polynucleotides which comprise at least two of the polynucleotides specified hereinabove, i.e. polynucleotides having a nucleotide sequence which contains at least two of the mutations comprised by the above polynucleotides or listed in Table 2 below. This allows the study of synergistic effects of said mutations in the TPMT gene and/or a polypeptide encoded by said polynucleotide on the pharmacological profile of drugs in patients who bear such mutant forms of the gene or similar mutant forms that can be mimicked by the above described proteins. It is expected that the analysis of said synergistic effects provides deeper insights into the onset of TPMT dysfunctions or dysregulations or diseases related to altered TPMT activity as described supra. From said deeper insight the development of diagnostic and pharmaceutical compositions related to TPMT dysfunctions or dysregulations or diseases related to impaired S-methylation of substrates, e.g. drugs such as thiopurines will greatly benefit.

As is evident to the person skilled in the art, the genetic knowledge deduced from the present invention can now be used to exactly and reliably characterize the genotype of a patient. Advantageously, diseases or a prevalence for a disease which are associated with TPMT dysfunction or dysregulation, such as thiopurine-induced toxicity, e.g. myelosupression (pancytopenia, leucopenia, thrombocytopenia, anemia) in the treatment of acute lymphoblastic leukaemia (ALL), autoimmune disorders, inflammatory bowel disease and organ transplant recipients referred to herein can be predicted and preventive or therapeutical measures can be applied accordingly. Moreover in accordance with the foregoing, in cases where a given drug takes an unusual effect, a suitable individual therapy can be designed based on the knowledge of the individual genetic makeup of a subject with respect to the polynucleotides of the invention and improved therapeutics can be developed as will be further discussed below.

In general, the TPMT “status”, defined by the expression level and activity of the TPMT protein, can be not only altered in many disease or disorders including thiopurine-induced toxicity (e.g. end stage renal failure), but can also be variable in normal tissue, due to genetic variations/polymorphisms. The identification of polymorphisms associated with altered TPMT expression and/or activity is important for the prediction of drug response and dosing and subsequently for the prediction of therapy outcome, including side effects of medications medications (thiopurine-induced toxicity, e.g. myelosupression (pancytopenia, leucopenia, thrombocytopenia, anemia)). Therefore, analysis of TPMT variations indicative of TPMT function, is a valuable tool for therapy with drugs, which are substrates of TPMT and has, thanks to the present invention, now become possible.

The present invention also relates to a method for selecting a suitable therapy to prevent TPMT associated diseases as indicated above, wherein said method comprises:
(a) determining the presence or absence of a variant allele referred to above in the genome of a subject in a sample obtained from said subject; and
(b) selecting a suitable therapy for said subject based on the results obtained in (a).

The definitions and explanations of the terms made above apply mutatis mutandis to the above method.

The term “suitable therapy” as used herein means that a TPMT substrate according to the invention is selected and said TPMT substrate being administered in a certain dosage to a subject, wherein said TPMT substrate and said dosage are selected based on the knowledge of the presence or absence of the variant allele referred to in accordance with the invention. Preferably said substrate are aromatic and heterocyclic compounds. Most preferably said substrates are thiopurines such as 6-mercaptopurine, 6-thioguanine or azothioprine. Said TPMT substrate and said dosage of the substrate are selected in a way that on one hand they are most effective in treating diseases such as acute lymphoblastic leukaemia (ALL), autoimmune disorders, inflammatory bowel disease, rheumatoid arthritis or organ transplant rejections, on the other hand they do not cause toxic or undesirable side effects.

As is evident from the above, a prerequisite for selecting a suitable therapy is the knowledge of the presence or absence of a variant allele referred to in accordance with the invention. Therefore, the method of the present invention encompasses the determination of the presence or absence of said variant alleles in a sample which has been obtained from said subject. The sample which is obtained by the subject comprises biological material which is suitable for the determination of the presence or absence of said variant alleles, such as isolated cells or tissue. Methods for the determination of the presence or absence of the variant alleles of the invention comprise those methods referred to herein below.

Thanks to the method of the present invention, it is possible to efficiently select a suitable therapy for a subject, preferably a human, suffering from diseases such as acute lymphoblastic leukaemia (ALL), autoimmune disorders, inflammatory bowel disease, rheumatoid arthritis or organ transplant rejections. Thereby, mistreatment of patients based on wrong dosages of TPMT substrates and the results thereof, such as severe and potentially fatal hematopoietic toxicity (e.g., pancytopenia), caused by the accumulation of cytotoxic metabolites or treatment failure due to insufficient accumulation of the active compounds after treatment with standard doses of thiopurines can be efficiently avoided. Furthermore, patients that are at high risk of developing toxic reactions to treatment with a TPMT substrate, for instance hematopoietic toxicity, can be excluded from therapy, or dosage can be adjusted according to the individual's genetic makeup prior to the onset of drug therapy. Also, in cases wherein inhibitors for the mentioned TPMT gene product, preferably sulfasalazine or olsalazine, are applied in genetically defined patient subpopulations, adverse effects can be avoided and the optimal drug level can be reached faster without time-consuming and expensive drug monitoring-based dose finding. This can reduce costs of medical treatment and indirect costs of disease (e.g. shorter time and less frequent hospitalization of patients).

Accordingly, the present invention also relates to a method of using a TPMT substrate to treat a patient suffering from diseases which comprises
(a) determining if the patient has one or more variant alleles of the present invention;
(b) in a patient having one or more of such variant alleles, administering to the patient an amount of the TPMT substrate which is sufficient to treat a patient having such variant alleles which amount is decreased in comparison to the amount that is administered without regard to the patient's alleles in the TPMT gene.

The term “the amount that is administered without regard to the patient's alleles in the TPMT gene” refers to the amount of TPMT substrate that is administered to a patient having normal TPMT activity, according to Mosby's GenRx, The complete reference for generic and brand drugs, 9th edition, Mosby's Inc. St. Louis (1999) and Martindale, The complete drug reference 33th edition, Pharmaceutical Press London (2002), however, without having regard to the patient's alleles in the TPMT gene. TPMT activity can be determined by assays described in the art and referred to in the examples such as Kroplin, Eur. J. Clin. Pharmacol. 55 (1999), 285-91; Kroplin, Eur. J. Clin. Pharmacol. 54 (1998), 265-71; Weinshilboum, Am. J. Hum. Genet. 32 (1980), 651-62. Preferably the TPMT substrates are aromatic and heterocyclic sulfhydryl compounds. Most preferably said TPMT substrates are thiopurines such as 6-mercaptopurine, 6-thioguanine and azathioprine.

In a preferred embodiment of the above method, the disease is acute lymphoblastic leukaemia (ALL), autoimmune disorders, inflammatory bowel disease and organ transplantation.

In another preferred embodiment of the method, the one or more variant alleles of the invention result in the patient expressing low activity of the TPMT gene product, whereby the amount of the TPMT substrate administered to the patient is decreased to reduce toxicity.

The invention further relates to a method for determining whether a patient is at increased risk for a toxic reaction to treatment with a TPMT substrate which comprises determining if the patient has one or more variant alleles of the TMPT gene according to the invention. In this case, the amount of the TPMT substrate administered to the patient is preferably decreased to reduce toxicity.

Finally, the polynucleotides and polypeptides referred to in accordance with the present invention are also useful as forensic markers, which improve the identification of subjects which have been murdered or killed by, for example a crime of violence or any other violence and can not be identified by the well known conventional forensic methods. The application of forensic methods based on the detection of the polymorphisms comprised by the polynucleotides of this invention in the genome of a subject are particularly well suited in cases where a (dead) body is disfigured in a severe manner such as identification by other body characteristics such as the features of the face is not possible. This is the case, for example, for corpses found in water which are usually entirely disfigured. Advantageously, methods which are based on the provision of the polynucleotides of the invention merely require a minimal amount of tissue or cells in order to be carried out. Said tissues or cells may be blood droplets, hair roots, epidermal scales, salivia droplets, sperms etc. Since only such a minimal amount of tissue or cells is required for the identification of a subject, the polymorphism comprised by the polynucleotides of this invention can also be used as forensic markers in order to proof someone guilty for a crime, such as a violation or a ravishment. Moreover, the polymorphisms comprised by the polynucleotides of this invention can be used to proof paternity. In accordance with the forensic methods referred herein the presence or absence of the polynucleotides of the invention is determined and compared with a reference sample which is unambiguously derived from the subject to be identified. The forensic methods which require detection of the presence or absence of the polynucleotides of this invention in a sample of a subject the polymorphisms comprised by the polynucleotides of this invention can be for example PCR-based techniques which are particularly well suited in cases where only minimal amount of tissue or cells is available as forensic samples. On the other hand, where enough tissue or cells is available, hybridization based techniques may be performed in order to detect the presence or absence of a polynucleotide of this invention. These techniques are well known by the person skilled in the art and can be adopted to the individual purposes referred to herein without further ado. In conclusion, thanks to the present invention forensic means which allow improved and reliable predictions as regards the aforementioned aspects are now available.

In line with the foregoing, preferably, the polynucleotide of the present invention is associated with a TPMT associated disease. Preferably, said disease is thiopurine-induced toxicity. More preferably, said thiopurine-induced toxicity is accompanied by myelosuppression (pancytopenia) and gastrointestinal disturbances. Most preferably, by leucopenia, thrombocytopenia, anemia.

The terms “myelosuppression”, “pancytopenia”, “leucopenia”, “thrombocytopenia”, or “anemia” used herein are very well known and characterized in the art. Several variants of thereof exist and are comprised by said term as meant in accordance with the invention. For a detailed list of symptoms which are indicative for myelosuppression, pancytopenia, leucopenia, thrombocytopenia or anemia it is referred to text book knowledge, e.g. Pschyrembel or Stedman.

In a further embodiment the present invention relates to a polynucleotide which is DNA or RNA.

The polynucleotide of the invention may be, e.g., DNA, cDNA, genomic DNA, RNA or synthetically produced DNA or RNA or a recombinantly produced chimeric nucleic acid molecule comprising any of those polynucleotides either alone or in combination. Preferably said polynucleotide is part of a vector, particularly plasmids, cosmids, viruses and bacteriophages used conventionally in genetic engineering that comprise a polynucleotide of the invention. Such vectors may comprise further genes such as marker genes which allow for the selection of said vector in a suitable host cell and under suitable conditions.

The invention furthermore relates to a gene comprising the polynucleotide of the invention.

It is well known in the art that genes comprise structural elements which encode an amino acid sequence as well as regulatory elements which are involved in the regulation of the expression of said genes. Structural elements are represented by exons which may either encode an amino acid sequence or which may encode for RNA which is not encoding an amino acid sequence but is nevertheless involved in RNA function, e.g. by regulating the stability of the RNA or the nuclear export of the RNA. Regulatory elements of a gene may comprise promoter elements or enhancer elements both of which could be involved in transcriptional control of gene expression. It is very well known in the art that a promoter is to be found upstream of the structural elements of a gene. Regulatory elements such as enhancer elements, however, can be found distributed over the entire locus of a gene. Said elements could be reside, e.g., in introns, regions of genomic DNA which separate the exons of a gene. Promoter or enhancer elements correspond to polynucleotide fragments which are capable of attracting or binding polypeptides involved in the regulation of the gene comprising said promoter or enhancer elements. For example, polypeptides involved in regulation of said gene comprise the so called transcription factors.

In addition, besides their function in transcriptional control and control of proper RNA processing and/or stability, regulatory elements of a gene could be also involved in the control of genetic stability of a gene locus. Said elements control, e.g., recombination events or serve to maintain a certain structure of the DNA or the arrangement of DNA in a chromosome.

Therefore, single nucleotide polymorphisms can occur in exons of a gene which encode an amino acid sequence as discussed supra as well as in regulatory regions which are involved in the above discussed process. The analysis of the nucleotide sequence of a gene locus in its entirety including, e.g., introns is in light of the above desirable. The polymorphisms comprised by the polynucleotides of the present invention can influence the expression level of TPMT protein via mechanisms involving enhanced or reduced transcription of the TPMT gene, stabilization of the gene's RNA transcripts and alteration of the processing of the primary RNA transcripts.

Therefore, in a furthermore preferred embodiment of the gene of the invention a nucleotide substitution results in altered expression of the variant gene compared to the corresponding wild type gene.

In another embodiment the present invention relates to a vector comprising the polynucleotide of the invention or the gene of the invention.

Said vector may be, for example, a phage, plasmid, viral or retroviral vector. Retroviral vectors may be replication competent or replication defective. In the latter case, viral propagation generally will occur only in complementing host/cells.

The polynucleotides or genes of the invention may be joined to a vector containing selectable markers for propagation in a host. Generally, a plasmid vector is introduced in a precipitate such as a calcium phosphate precipitate, or in a complex with a charged lipid or in carbon-based clusters. Should the vector be a virus, it may be packaged in vitro using an appropriate packaging cell line prior to application to host cells.

In a more preferred embodiment of the vector of the invention the polynucleotide is operatively linked to expression control sequences allowing expression in prokaryotic or eukaryotic cells or isolated fractions thereof.

Expression of said polynucleotide comprises transcription of the polynucleotide, preferably into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pCDM8, pRc/CMV, pcDNA1, pcDNA3 (In-vitrogene), pSPORT1 (GIBCO BRL). Preferably, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Alternatively, the polynucleotides and vectors of the invention can be reconstituted into liposomes for delivery to target cells.

The term “isolated fractions thereof” refers to fractions of eukaryotic or prokaryotic cells or tissues which are capable of transcribing or transcribing and translating RNA from the vector of the invention. Said fractions comprise proteins which are required for transcription of RNA or transcription of RNA and translation of said RNA into a polypeptide. Said isolated fractions may be, e.g., nuclear and cytoplasmic fractions of eukaryotic cells such as of reticulocytes.

The present invention furthermore relates to a host cell genetically engineered with the polynucleotide of the invention, the gene of the invention or the vector of the invention. Said host cell may be a prokaryotic or eukaryotic cell; see supra. The polynucleotide or vector of the invention which is present in the host cell may either be integrated into the genome of the host cell or it may be maintained extrachromosomally. In this respect, it is also to be understood that the recombinant DNA molecule of the invention can be used for “gene targeting” and/or “gene replacement”, for restoring a mutant gene or for creating a mutant gene via homologous recombination; see for example Mouellic, Proc. Natl. Acad. Sci. USA, 87 (1990), 4712-4716; Joyner, Gene Targeting, A Practical Approach, Oxford University Press.

The host cell can be any prokaryotic or eukaryotic cell, such as a bacterial, insect, fungal, plant, animal, mammalian or, preferably, human cell. Preferred fungal cells are, for example, those of the genus Saccharomyces, in particular those of the species S. cerevisiae. The term “prokaryotic” is meant to include all bacteria which can be transformed or transfected with a polynucleotide for the expression of a variant polypeptide of the invention. Prokaryotic hosts may include gram negative as well as gram positive bacteria such as, for example, E. coli, S. typhimurium, Serratia marcescens and Bacillus subtilis. A polynucleotide coding for a mutant form of variant polypeptides of the invention can be used to transform or transfect the host using any of the techniques commonly known to those of ordinary skill in the art. Methods for preparing fused, operably linked genes and expressing them in bacteria or animal cells are well-known in the art (Sambrook, supra). The genetic constructs and methods described therein can be utilized for expression of variant polypeptides of the invention in, e.g., prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. The transformed prokaryotic hosts can be grown in fermentors and cultured according to techniques known in the art to achieve optimal cell growth. The proteins of the invention can then be isolated from the grown medium, cellular lysates, or cellular membrane fractions. The isolation and purification of the microbially or otherwise expressed polypeptides of the invention may be by any conventional means such as, for example, preparative chromatographic separations and immunological separations such as those involving the use of monoclonal or polyclonal antibodies.

Thus, in a further embodiment the invention relates to a method for producing a molecular variant TPMT polypeptide or fragment thereof comprising culturing the above described host cell; and recovering said protein or fragment from the culture.

In another embodiment the present invention relates to a method for producing cells capable of expressing a molecular variant TPMT polypeptide comprising genetically engineering cells with the polynucleotide of the invention, the gene of the invention or the vector of the invention.

The cells obtainable by the method of the invention can be used, for example, to test drugs according to the methods described in D. L. Spector, R. D. Goldman, L. A. Leinwand, Cells, a Lab manual, CSH Press 1998. Furthermore, the cells can be used to study known drugs and unknown derivatives thereof for their ability to complement the deficiency caused by mutations in the TPMT gene. For these embodiments the host cells preferably lack a wild type allele, preferably both alleles of the TPMT gene and/or have at least one mutated from thereof. Ideally, the gene comprising an allele as comprised by the polynucleotides of the invention could be introduced into the wild type locus by homologous replacement. Alternatively, strong overexpression of a mutated allele over the normal allele and comparison with a recombinant cell line overexpressing the normal allele at a similar level may be used as a screening and analysis system. The cells obtainable by the above-described method may also be used for the screening methods referred to herein below.

Furthermore, the invention relates to a polypeptide or fragment thereof encoded by the polynucleotide of the invention, the gene of the invention or obtainable by the method described above or from cells produced by the method described above.

In this context it is also understood that the variant polypeptide of the invention can be further modified by conventional methods known in the art. By providing said variant proteins according to the present invention it is also possible to determine the portions relevant for their biological activity or inhibition of the same. The terms “polypeptide” and “protein” as used herein are exchangeable. Moreover, what is comprised by said terms is standard textbook knowledge.

The present invention furthermore relates to an antibody which binds specifically to the polypeptide of the invention.

Advantageously, the antibody specifically recognizes or binds an epitope containing one or more amino acid substitution(s) as defined above. Antibodies against the variant polypeptides of the invention can be prepared by well known methods using a purified protein according to the invention or a (synthetic) fragment derived therefrom as an antigen. Monoclonal antibodies can be prepared, for example, by the techniques as originally described in Köhler and Milstein, Nature 256 (1975), 495, and Galfré, Meth. Enzymol. 73 (1981), 3, which comprise the fusion of mouse myeloma cells to spleen cells derived from immunized mammals. In a preferred embodiment of the invention, said antibody is a monoclonal antibody, a polyclonal antibody, a single chain antibody, human or humanized antibody, primatized, chimerized or fragment thereof that specifically binds said peptide or polypeptide also including bispecific antibody, synthetic antibody, antibody fragment, such as Fab, Fv or scFv fragments etc., or a chemically modified derivative of any of these. Furthermore, antibodies or fragments thereof to the aforementioned polypeptides can be obtained by using methods which are described, e.g., in Harlow and Lane “Antibodies, A Laboratory Manual”, CSH Press, Cold Spring Harbor, 1988. These antibodies can be used, for example, for the immunoprecipitation and immunolocalization of the variant polypeptides of the invention as well as for the monitoring of the presence of said variant polypeptides, for example, in recombinant organisms, and for the identification of compounds interacting with the proteins according to the invention. For example, surface plasmon resonance as employed in the BIAcore system can be used to increase the efficiency of phage antibodies which bind to an epitope of the protein of the invention (Schier, Human Antibodies Hybridomas 7 (1996), 97-105; Malmborg, J. Immunol. Methods 183 (1995), 7-13).

In a preferred embodiment the antibody of the present invention specifically recognizes an epitope containing one or more amino acid substitution(s) resulting from a nucleotide exchange as defined supra.

Antibodies which specifically recognize modified amino acids such as phospho-Tyrosine residues are well known in the art. Similarly, in accordance with the present invention antibodies which specifically recognize even a single amino acid exchange in an epitope may be generated by the well known methods described supra.

In light of the foregoing, in a more preferred embodiment the antibody of the present invention is monoclonal or polyclonal.

The invention also relates to a transgenic non-human animal comprising at least one polynucleotide of the invention, the gene of the invention or the vector of the invention as described supra.

The present invention also encompasses a method for the production of a transgenic non-human animal comprising introduction of a polynucleotide or vector of the invention into a germ cell, an embryonic cell, stem cell or an egg or a cell derived therefrom. The non-human animal can be used in accordance with the method of the invention described below and may be a non-transgenic healthy animal, or may have a disease or disorder, preferably a disease caused by at least one mutation in the gene of the invention. Such transgenic animals are well suited for, e.g., pharmacological studies of drugs in connection with variant forms of the above described variant polypeptides since these polypeptides or at least their functional domains are conserved between species in higher eukaryotes, particularly in mammals. Production of transgenic embryos and screening of those can be performed, e.g., as described by A. L. Joyner Ed., Gene Targeting, A Practical Approach (1993), Oxford University Press. The DNA of the embryos can be analyzed using, e.g., Southern blots with an appropriate probe or based on PCR techniques.

A transgenic non-human animal in accordance with the invention may be a transgenic mouse, rat, hamster, dog, monkey, rabbit, pig, frog, nematode such as Caenorhabditis elegans, fruitfly such as Drosophila melanogaster or fish such as torpedo fish or zebrafish comprising a polynucleotide or vector of the invention or obtained by the method described above, preferably wherein said polynucleotide or vector is stably integrated into the genome of said non-human animal, preferably such that the presence of said polynucleotide or vector leads to the expression of the variant polypeptide of the invention. It may comprise one or several copies of the same or different polynucleotides or genes of the invention. This animal has numerous utilities, including as a research model for cardiovascular research and therefore, presents a novel and valuable animal in the development of therapies, treatment, etc. for diseases caused by cardiovascular diseases. Accordingly, in this instance, the mammal is preferably a laboratory animal such as a mouse or rat.

Thus, in a preferred embodiment the transgenic non-human animal of the invention is a mouse, a rat or a zebrafish.

Numerous reports revealed that said animals are particularly well suited as model organisms for the investigation of the drug metabolism and its deficiencies or cancer. Advantageously, transgenic animals can be easily created using said model organisms, due to the availability of various suitable techniques well known in the art.

The invention also relates to a solid support comprising one or a plurality of the polynucleotide, the gene, the vector, the polypeptide, the antibody or the host cell of the invention in immobilized form.

The term “solid support” as used herein refers to a flexible or non-flexible support that is suitable for carrying said immobilized targets. Said solid support may be homogenous or inhomogeneous. For example, said solid support may consist of different materials having the same or different properties with respect to flexibility and immobilization, for instance, or said solid support may consist of one material exhibiting a plurality of properties also comprising flexibility and immobilization properties. Said solid support may comprise glass-, polypropylene- or silicon-chips, membranes oligonucleotide-conjugated beads or bead arrays.

The term “immobilized” means that the molecular species of interest is fixed to a solid support, preferably covalently linked thereto. This covalent linkage can be achieved by different means depending on the molecular nature of the molecular species. Moreover, the molecular species may be also fixed on the solid support by electrostatic forces, hydrophobic or hydrophilic interactions or Van-der-Waals forces. The above described physico-chemical interactions typically occur in interactions between molecules. For example, biotinylated polypeptides may be fixed on a avidin-coated solid support due to interactions of the above described types. Further, polypeptides such as antibodies, may be fixed on an antibody coated solid support. Moreover, the immobilization is dependent on the chemical properties of the solid support. For example, the nucleic acid molecules can be immobilized on a membrane by standard techniques such as UV-crosslinking or heat.

In a preferred embodiment of the invention said solid support is a membrane, a glass- or poylpropylene- or silicon-chip, are membranes oligonucleotide-conjugated beads or a bead array, which is assembled on an optical filter substrate.

Moreover, the present invention relates to an in vitro method for identifying a polymorphism said method comprising the steps of:
(a) isolating a polynucleotide or the gene of the invention from a plurality of subgroups of individuals, wherein one subgroup has no prevalence for a TPMT associated disease and at least one or more further subgroup(s) do have prevalence for a TPMT associated disease; and
(b) identifying a polymorphism by comparing the nucleic acid sequence of said polynucleotide or said gene of said one subgroup having no prevalence for a TPMT associated disease with said at least one or more further subgroup(s) having a prevalence for a TPMT associated disease.

The term “prevalence” as used herein means that individuals are be susceptible for one or more disease(s) which are associated with TPMT dysfuntion or dysregulation or could already have one or more of said disease(s). Moreover, symptoms which are indicative for a prevalence for developing said diseases are very well known in the art and have been sufficiently described in standard textbooks such as Pschyrembel. Advantageously, polymorphisms according to the present invention which are associated with TPMT dysfunction or dysregulation or one or more disease(s) based thereon should be enriched in subgroups of individuals which have a prevalence for said diseases versus subgroups which have no prevalence for said diseases. Thus, the above described method allows the rapid and reliable detection of polymorphism which are indicative for one or more TPMT associated disease(s) or a susceptibility therefor. Advantageously, due to the phenotypic preselection a large number of individuals having no prevalence might be screened for polymorphisms in general. Thereby, a reference sequences comprising polymorphisms which do not correlate to one or more TPMT associated disease(s) can be obtained. Based on said reference sequences it is possible to efficiently and reliably determine the relevant polymorphisms.

In a further embodiment the present invention relates to a method for identifying and obtaining a pro-drug or a drug capable of modulating the activity of a molecular variant of a TPMT polypeptide comprising the steps of:
(a) contacting the polypeptide, the solid support of the invention, a cell expressing a molecular variant gene comprising a polynucleotide of the invention, the gene or the vector of the invention in the presence of components capable of providing a detectable signal in response to drug activity with a compound to be screened for pro-drug or drug activity; and
(b) detecting the presence or absence of a signal or increase or decrease of a signal generated from the pro-drug or the drug activity, wherein the absence, presence, increase or decrease of the signal is indicative for a putative pro-drug or drug.

The term “compound” in a method of the invention includes a single substance or a plurality of substances which may or may not be identical.

Said compound(s) may be chemically synthesized or produced via microbial fermentation but can also be comprised in, for example, samples, e.g., cell extracts from, e.g., plants, animals or microorganisms. Furthermore, said compounds may be known in the art but hitherto not known to be useful as an inhibitor, respectively. The plurality of compounds may be, e.g., added to the culture medium or injected into a cell or non-human animal of the invention.

If a sample containing (a) compound(s) is identified in the method of the invention, then it is either possible to isolate the compound from the original sample identified as containing the compound, in question or one can further subdivide the original sample, for example, if it consists of a plurality of different compounds, so as to reduce the number of different substances per sample and repeat the method with the subdivisions of the original sample. It can then be determined whether said sample or compound displays the desired properties, for example, by the methods described therein or in the literature (Spector et al., Cells manual; see supra). Depending on the complexity of the samples, the steps described above can be performed several times, preferably until the sample identified according to the method of the invention only comprises a limited number of or only one substance(s). Preferably said sample comprises substances of similar chemical and/or physical properties, and most preferably said substances are identical. The methods of the present invention can be easily performed and designed by the person skilled in the art, for example in accordance with other cell based assays described in the prior art or by using and modifying the methods as described herein. Furthermore, the person skilled in the art will readily recognize which further compounds may be used in order to perform the methods of the invention, for example, enzymes, if necessary, that convert a certain compound into a precursor. Such adaptation of the method of the invention is well within the skill of the person skilled in the art and can be performed without undue experimentation.

Compounds which can be used in accordance with the present invention include peptides, proteins, nucleic acids, antibodies, small organic compounds, ligands, peptidomimetics, PNAs and the like. Said compounds may act as agonists or antagonists of the inveniton. Said compounds can also be functional derivatives or analogues of known drugs. Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U.S.A. and Organic Synthesis, Wiley, New York, USA. Furthermore, said derivatives and analogues can be tested for their effects according to methods known in the art or as described. Furthermore, peptide mimetics and/or computer aided design of appropriate drug derivatives and analogues can be used, for example, according to the methods described below. Such analogs comprise molecules may have as the basis structure of known TPMT substrates, e.g. thiopurine drugs (azathioprine, 6-mercaptopurine, 6-thioguanine) and/or inhibitors, e.g. aminosalicylic acid derivates, sulfasalazine; see infra.

Appropriate computer programs can be used for the identification of interactive sites of a putative inhibitor and the polypeptides of the invention by computer assistant searches for complementary structural motifs (Fassina, Immunomethods 5 (1994), 114-120). Further appropriate computer systems for the computer aided design of protein and peptides are described in the prior art, for example, in Berry, Biochem. Soc; Trans. 22 (1994), 1033-1036; Wodak, Ann. N.Y. Acad. Sci. 501 (1987), 1-13; Pabo, Biochemistry 25 (1986), 5987-5991. The results obtained from the above-described computer analysis can be used in combination with the method of the invention for, e.g., optimizing known inhibitors, analogs, antagonists or agonists. Appropriate peptidomimetics and other inhibitors can also be identified by the synthesis of peptidomimetic combinatorial libraries through successive chemical modification and testing the resulting compounds, e.g., according to the methods described herein. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Furthermore, the three-dimensional and/or crystallographic structure of said compounds and the polypeptides of the invention can be used for the design of peptidomimetic drugs (Rose, Biochemistry 35 (1996), 12933-12944; Rutenber, Bioorg. Med. Chem. 4 (1996), 1545-1558). It is very well known how to obtain said compounds, e.g. by chemical or biochemical standard techniques. Thus, also comprised by the method of the invention are means of making or producing said compounds. In summary, the present invention provides methods for identifying and obtaining compounds which can be used in specific doses for the treatment of specific forms of TPMT associated diseases, e.g. dysfunctions or dysregulations of the drug metabolism such as myelosuppression (pancytopenia, leucopenia, thrombocytopenia, anemia).

The above definitions apply mutatis mutandis to all of the methods described in the following.

In a further embodiment the present invention relates to a method for identifying and obtaining an inhibitor of the activity of a molecular variant of a TPMT polypeptide comprising the steps of:
(a) contacting the protein, the solid support of the invention or a cell expressing a molecular variant gene comprising a polynucleotide or the gene or the vector of the invention in the presence of components capable of providing a detectable signal in response to drug activity with a compound to be screened for inhibiting activity; and
(b) detecting the presence or absence of a signal or increase or decrease of a signal generating from the inhibiting activity, wherein the absence or decrease of the signal is indicative for a putative inhibitor.

In a preferred embodiment of the method of the invention said cell is a cell, obtained by the method of the invention or can be obtained from the transgenic non-human animal as described supra.

In a still further embodiment the present invention relates to a method of identifying and obtaining a pro-drug or drug capable of modulating the activity of a molecular variant of a TPMT polypeptide comprising the steps of:
(a) contacting the host cell, the cell obtained by the method of the invention, the polypeptide or the solid support of the invention with the first molecule known to be bound by a TPMT polypeptide to form a first complex of said polypeptide and said first molecule;
(b) contacting said first complex with a compound to be screened, and
(c) measuring whether said compound displaces said first molecule from said first complex.

Advantageously, in said method said measuring step comprises measuring the formation of a second complex of said protein and said inhibitor candidate. Preferably, said measuring step comprises measuring the amount of said first molecule that is not bound to said protein.

In a particularly preferred embodiment of the above-described method of said first molecule is a agonist or antagonist or a substrate and/or a inhibitor and/or a modulator of the polypeptide of the invention, e.g., with a radioactive or fluorescent label.

In a still another embodiment the present invention relates to a method of identifying and obtaining an inhibitor capable of modulating the activity of a molecular variant of a TPMT polypeptide comprising the steps of:
(a) contacting the host cell or the cell obtained by the method of the invention, the protein or the solid support of the invention with the first molecule known to be bound by the TPMT polypeptide to form a first complex of said protein and said first molecule;
(b) contacting said first complex with a compound to be screened, and
(c) measuring whether said compound displaces said first molecule from said first complex.

In a preferred embodiment of the method of the invention said measuring step comprises measuring the formation of a second complex of said protein and said compound.

In another preferred embodiment of the method of the invention said measuring step comprises measuring the amount of said first molecule that is not bound to said protein.

In a more preferred embodiment of the method of the invention said first molecule is labeled.

The invention furthermore relates to a method for the production of a pharmaceutical composition comprising the steps of the method as described supra; and the further step of formulating the compound identified and obtained or a derivative thereof in a pharmaceutically acceptable form.

The therapeutically useful compounds identified according to the methods of the invention can be formulated and administered to a patient as discussed above. For uses and therapeutic doses determined to be appropriate by one skilled in the art and for definitions of the term “pharmaceutical composition” see infra.

Furthermore, the present invention encompasses a method for the preparation of a pharmaceutical composition comprising the steps of the above-described methods; and formulating a drug or pro-drug in the form suitable for therapeutic application and preventing or ameliorating the disorder of the subject diagnosed in the method of the invention.

Drugs or pro-drugs after their in vivo administration are metabolized in order to be eliminated either by excretion or by metabolism to one or more active or inactive metabolites (Meyer, J. Pharmacokinet. Biopharm. 24 (1996), 449-459). Thus, rather than using the actual compound or inhibitor identified and obtained in accordance with the methods of the present invention a corresponding formulation as a pro-drug can be used which is converted into its active in the patient. Precautionary measures that may be taken for the application of pro-drugs and drugs are described in the literature; see, for review, Ozama, J. Toxicol. Sci. 21 (1996), 323-329).

In a preferred embodiment of the method of the present invention said drug or prodrug is a derivative of a medicament as defined hereinafter.

The present invention also relates to a method of diagnosing a TPMT associated disease or a susceptibility therefor comprising determining the presence of a polynucleotide or the gene of the invention in a sample from a subject.

In accordance with this embodiment of the present invention, the method of testing the status of a disorder or susceptibility to such a disorder can be effected by using a polynucleotide gene or nucleic acid of the invention, e.g., in the form of a Southern or Northern blot or in situ analysis. Said nucleic acid sequence may hybridize to a coding region of either of the genes or to a non-coding region, e.g. intron. In the case that a complementary sequence is employed in the method of the invention, said nucleic acid molecule can again be used in Northern blots. Additionally, said testing can be done in conjunction with an actual blocking, e.g., of the transcription of the gene and thus is expected to have therapeutic relevance. Furthermore, a primer or oligonucleotide can also be used for hybridizing to one of the above mentioned TPMT gene or corresponding mRNAs. The nucleic acids used for hybridization can, of course, be conveniently labeled by incorporating or attaching, e.g., a radioactive or other marker. Such markers are well known in the art. The labeling of said nucleic acid molecules can be effected by conventional methods.

Additionally, the presence or expression of variant TPMT gene can be monitored by using a primer pair that specifically hybridizes to either of the corresponding nucleic acid sequences and by carrying out a PCR reaction according to standard procedures. Specific hybridization of the above mentioned probes or primers preferably occurs at stringent hybridization conditions. The term “stringent hybridization conditions” is well known in the art; see, for example, Sambrook et al., “Molecular Cloning, A Laboratory Manual” second ed., CSH Press, Cold Spring Harbor, 1989; “Nucleic Acid Hybridisation, A Practical Approach”, Hames and Higgins eds., IRL Press, Oxford, 1985. Furthermore, the mRNA, cRNA, cDNA or genomic DNA obtained from the subject may be sequenced to identify mutations which may be characteristic fingerprints of mutations in the polynucleotide or the gene of the invention. The present invention further comprises methods wherein such a fingerprint may be generated by RFLPs of DNA or RNA obtained from the subject optionally the DNA or RNA may be amplified prior to analysis, the methods of which are well known in the art. RNA fingerprints may be performed by, for example, digesting an RNA sample obtained from the subject with a suitable RNA-Enzyme, for example RNase T₁, RNase T₂ or the like or a ribozyme and, for example, electrophoretically separating and detecting the RNA fragments as described above.

Further modifications of the above-mentioned embodiment of the invention can be easily devised by the person skilled in the art, without any undue experimentation from this disclosure; see, e.g., the examples. An additional embodiment of the present invention relates to a method wherein said determination is effected by employing an antibody of the invention or fragment thereof. The antibody used in the method of the invention may be labeled with detectable tags such as a histidine flags or a biotin molecule.

The invention relates to a method of diagnosing a TMPT associated disease or a susceptibility therefor comprising determining the presence of a polypeptide or the antibody of the invention in a sample from a subject.

In a preferred embodiment of the above described method said disorder is a thiopurine-induced toxicity, preferably, myelosuppression (pancytopenia) and gastrointestinal disturbances, more preferably, leucopenia, thrombocytopenia, anemia.

In another preferred embodiment the method described above comprises PCR based techniques, RFLP-based techniques, DNA sequencing-based techniques, hybridization techniques, Single strand conformational polymorphism (SSCP), denaturating gradient gel electrophoresis (DGGE), mismatch cleavage detection, heteroduplex analysis, techniques based on mass spectroscopy, HPLC-based techniques, primer extension-based techniques, and 5′-nuclease assay-based techniques.

Moreover, the invention relates to a method of detection of the polynucleotide or the gene of the invention in a sample comprising the steps of
(a) contacting the solid support described supra with the sample under conditions allowing interaction of the polynucleotide or the gene of the invention with the immobilized targets on a solid support and;
(b) determining the binding of said polynucleotide or said gene to said immobilized targets on a solid support.

The invention also relates to an in vitro method for diagnosing a disease comprising the steps of the method described supra, wherein binding of said polynucleotide or gene to said immobilized targets on said solid support is indicative for the presence or the absence of said disease or a prevalence for said disease.

In a preferred embodiment of the above described method said disorder is a thiopurine-induced toxicity, preferably, myelosuppression (pancytopenia) and gastrointestinal disturbances, more preferably, leucopenia, thrombocytopenia, anemia.

The invention furthermore relates to a diagnostic composition comprising the polynucleotide, the gene, the vector, the polypeptide or the antibody of the invention.

In addition, the invention relates to a pharmaceutical composition comprising the polynucleotide, the gene, the vector, the polypeptide or the antibody of the invention. These pharmaceutical compositions comprising, e.g., the antibody may conveniently be administered by any of the routes conventionally used for drug administration, for instance, orally, topically, parenterally or by inhalation. Acceptable salts comprise acetate, methylester, HCl, sulfate, chloride and the like. The compounds may be administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof. The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax.

The dosage regimen will be determined by the attending physician and other clinical factors; preferably in accordance with any one of the above described methods. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment. Furthermore, the use of pharmaceutical compositions which comprise antisense-oligonucleotides which specifically hybridize to RNA encoding mutated versions of the polynucleotitde or gene according to the invention or which comprise antibodies specifically recognizing a mutated polypeptide of the invention but not or not substantially the functional wild-type form is conceivable in cases in which the concentration of the mutated form in the cells should be reduced.

Thanks to the present invention the particular drug selection, dosage regimen and corresponding patients to be treated can be determined in accordance with the present invention. The dosing recommendations will be indicated in product labeling by allowing the prescriber to anticipate dose adjustments depending on the considered patient group, with information that avoids prescribing the wrong drug to the wrong patients at the wrong dose.

A gene encoding a functional and expressible polypeptide of the invention can be introduced into the cells which in turn produce the protein of interest. Gene therapy, which is based on introducing therapeutic genes into cells by ex-vivo or in-vivo techniques is one of the most important applications of gene transfer. Suitable vectors and methods for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Isner, Lancet 348 (1996), 370-374; Muhlhauser, Circ. Res. 77 (1995), 1077-1086; Wang, Nature Medicine 2 (1996), 714-716; WO94/29469; WO 97/00957 or Schaper, Current Opinion in Biotechnology 7 (1996), 635-640, and references cited therein. The gene may be designed for direct introduction or for introduction via liposomes, or viral vectors (e.g. adenoviral, retroviral) into the cell. Preferably, said cell is a germ line cell, embryonic cell, or egg cell or derived therefrom, most preferably said cell is a stem cell. As is evident from the above, it is preferred that in the use of the invention the nucleic acid sequence is operatively linked to regulatory elements allowing for the expression and/or targeting of the polypeptides of the invention to specific cells. Suitable gene delivery systems that can be employed in accordance with the invention may include liposomes, receptor-mediated delivery systems, naked DNA, and viral vectors such as herpes viruses, retroviruses, adenoviruses, and adeno-associated viruses, among others. Delivery of nucleic acids to a specific site in the body for gene therapy may also be accomplished using a biolistic delivery system, such as that described by Williams (Proc. Natl. Acad. Sci. USA 88 (1991), 2726-2729). Standard methods for transfecting cells with recombinant DNA are well known to those skilled in the art of molecular biology, see, e.g., WO 94/29469; see also supra. Gene therapy may be carried out by directly administering the recombinant DNA molecule or vector of the invention to a patient or by transfecting cells with the polynucleotide or vector of the invention ex vivo and infusing the transfected cells into the patient.

Accordingly, the present invention encompasses the use of the polynucleotide, the gene, the vector, the polypeptide or the antibody of the invention for the preparation of a diagnostic composition for diagnosing a TMPT associated disease or a susceptibility therefor.

Further, the present invention encompasses the use of the polynucleotide, the gene, the vector, the polypeptide or the antibody of the invention for the preparation of a pharmaceutical composition for treating a TMPT associated disease or a susceptibility therefor.

In a preferred embodiment of the use of the present invention said disease is thiopurine-induced toxicity, preferably myelosuppression (pancytopenia) and gastrointestinal disturbances, more preferably leucopenia, thrombocytopenia, anemia.

Finally, the present invention relates to a diagnostic kit for detection of a single nucleotide polymorphism comprising the polynucleotide, the gene, the vector, the polypeptide, the antibody, the host cell, the transgenic non-human animal or the solid support of the invention.

The kit of the invention may contain further ingredients such as selection markers and components for selective media suitable for the generation of transgenic cells and animals. The kit of the invention can be used for carrying out a method of the invention and could be, inter alia, employed in a variety of applications, e.g., in the diagnostic field or as research tool. The parts of the kit of the invention can be packaged individually in vials or other appropriate means depending on the respective ingredient or in combination in suitable containers or multicontainer units. Manufacture of the kit follows preferably standard procedures which are known to the person skilled in the art. The kit may be used for methods for detecting expression of a mutant form of the polypeptides, genes or polynucleotides in accordance with any one of the above-described methods of the invention, employing, for example, immunoassay techniques such as radioimmunoassay or enzymeimmunoassay or preferably nucleic acid hybridization and/or amplification techniques such as those described herein before and in the Examples as well as pharmacokinetic studies when using non-human transgenic animals of the invention.

The nucleic acid and amino acid sequences referred to herein by making reference to SEQ ID Nos are shown in the following tables 1 to 3:

TABLE 1
Primers used for PCR amplification

| | | | | |
|---|---|---|---|---|
| | Seq | | Seq | |
| | ID | | ID | |
| site | No | Forward | No | reverse |

| | | | | |
|---|---|---|---|---|
| Exon | 1 | TGTCCTCTGTGATATTCC | 2 | GTGGATGTTACACAGGA |
| 6 | | TCTGAGTTG | | GGAAGGAG |
| Exon | 3 | CCCTCTATTTAGTCATTT | 4 | GAATGGTATCCTCATAA |
| 5 | | GAAAAC | TACTC | |
| Exon | 5 | CTCCACACCCAGGTCCAC | 6 | AGGTCTCTGTAGTCAAA |
| 7 | | ACATT | | TCCTATA |
| Exon | 25 | ACTGCTAAGAATAATAGG | 26 | GCCACAGATGCACTGTG |
| 3 | | TTTTCATTTAGTTC | | ACTCGGGAG |
| Exon | 27 | TACCACTGACTGGGTGTG | 28 | CTCAATCCAGAAAGACT |
| 4 | | TGTCTGA | | TCATACCTGTT |
| Exon | 29 | AATCCCTGATGTCATTCT | 30 | CATCCATTACATTTTCA |
| 10 | | TCATAGTATTT | | GGCTTTAGCATAAT |

TABLE 2
Summary of SNPs in TPMT:

| | | | | | |
|---|---|---|---|---|---|
| | | | | Seq | Seq |
| | Varia- | | GenBank | ID No | ID No |
| Site | tion | Position | Acc. No. | Forward | reverse |

| | | | | | |
|---|---|---|---|---|---|
| exon3 | C>G | 488 | AF019364.1 | 9 | 10 |
| exon4 | G>A | 391 | AF019365.1 | 11 | 12 |
| exon5 | A>C | 463 | AF019366.1 | 7 | 8 |
| exon6 | G>A | 516 | AF019367.1 | 13 | 14 |
| exon7 | G>A | 1236 | AF019367.1 | 15 | 16 |
| exon10 | A>G | 679 | AF019369.1 | 17 | 18 |

| | | | |
|---|---|---|---|
| Seq | | Seq | |
| ID | | ID | |
| No | Forward | No | reverse |

| | | | |
|---|---|---|---|
| 7 | CCTGGAACCACAGTATTTAA | 8 | CCTTAAATACTGTGGTTCCA |
| | GG | GG | |
| 9 | TGCTTTTCATGAGGAACAAGG | 10 | CCTTGTTCCTCATGAAAAGCA |
| 11 | TCCTCTTTGCAGAAAAGCGGT | 12 | ACCGCTTTTCTGCAAAGAGGA |
| 13 | TCATTGTACTATTGCAGTATT | 14 | AATACTGCAATAGTACAATGA |
| 15 | CCAGGTGATCACAAATGGTAA | 16 | TTACCATTTGTGATCACCTGG |
| 17 | TCTTTTTGAAGAGTTATATCT | 18 | AGATATAACTCTTCAAAAAGA |

TABLE 3
TPMT amino acid exchanges caused by the SNPs

| | | | Seq | |
|---|---|---|---|---|
| | AA | Protein | ID | |
| Gene | change | Acc No | No | Protein mut |
| TPMT | Q42E | AAC51865.1 | 19 | KTAFHEEQGH |
| TPMT | G71R | AAC51865.1 | 20 | FFPLCRKAVEM |
| TPMT | K119T | AAC51865.1 | 21 | EIPGTTVFKSS |
| TPMT | C132Y | AAC51865.1 | 22 | NISLYYCSIFD |
| TPMT | R163H | AAC51865.1 | 23 | INPGDHKCYAD |
| TPMT | K238E | AAC51865.1 | 24 | DCLFEELYLLT |

The FIGURE illustrates the invention:

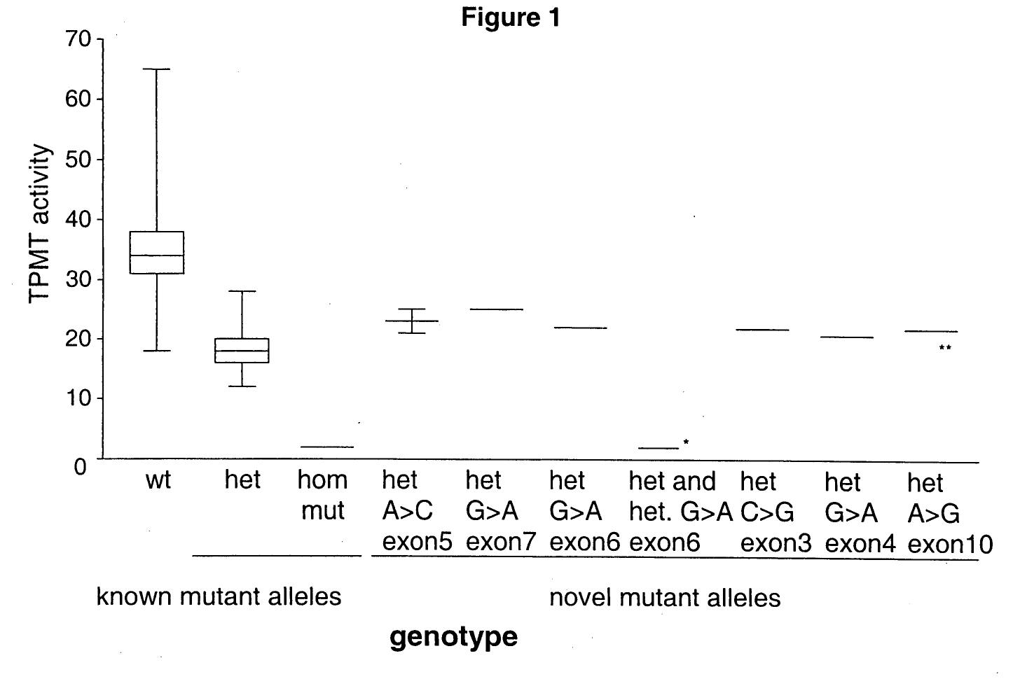

FIG. 1: Genotype-Phenotype correlation of TPMT in healthy blood donors (n=1200), in one child (*) treated by the acute lymphoblastic leukemia (ALL) protocol and in one patient with thiopurine treatment due to inflammatory bowel disease (**). In the case of reduced and very low TPMT activity, which could not be explained by known mutant alleles novel SNPs could be identified. (Wt: wildtype; het. mut: one mutant allele; hom. mut.: two mutant alleles)

The invention will now be described by reference to the following biological Examples which are merely illustrative and are not constructed as a limitation of the scope of the present invention.

### EXAMPLE 1

### Isolation of Genomic DNA and Generation and Purification of TPMT PCR Fragments

Blood samples were obtained from healthy blood donors, from one child with acute lymphoblastic leukemia (ALL) treated in the German multicentre study protocol BFM-ALL 2000 and from one patient with thiopurine treatment due to other causes (inflammatory bowel disease). Genomic DNA was isolated by standard procedures (QIAamp DNA Blood Mini Kit). Blood from all the individuals tested was obtained under consideration of all legal, medical and bureaucratical requirements. Specific oligonucleotide primers were applied to obtain defined DNA fragments containing specific parts of the TPMT gene by polymerase chain reaction (PCR). These specific oligonucleotide primers were designed to bind to sequence regions upstream and downstream of the various exons of the TPMT gene. The resulting DNA fragments did not contain codogenic parts alone but also sequences covering the intronic parts located at the exon-intron boundaries. Commercially synthesized oligonucleotide primer pairs that were purified by affinity chromatography were optimized for each of the six described exon fragments of the human TPMT gene. For exon 3, 4, 6, 7 and 10 published primers were used, while for exon 5 novel primers were designed. The sequence for each primer is listed in table 1.

Polymerase chain reactions were performed under conditions that were optimized for all three fragments. PCRs were carried out for all exons in a volume of 25 μl, containing 50 ng of genomic DNA, 20 pmol of forward and reverse primer (MWG Biotech), 200 μM dNTPs, 0.25 U of Taq Polymerase and 1× buffer supplied by the manufacturer (Perkin-Elmer). PCRs were initiated by a denaturation step at 94° C. for 5 min, followed by 25 cycles at 94° C. for 30 sec, 59° C. for 1 min and 70° C. for 30 sec; a final extension was performed at 70° C. for 7 min. All PCR reactions were performed on an MJ Research thermocycler (PTC-225).

The defined DNA fragments containing specific parts of the TPMT gene, exon as well as some intron sequences at the intron-exon boundaries were processed to remove non incorporated nucleotides and buffer components that might otherwise interfere with the subsequent determination of the individual TPMT genotype by direct cycle sequencing. For this purification, standard ion-exchange chromatography techniques were used (QIAquick PCR purification kit). For all fragments sufficient yields of purified TPMT fragments were subjected to direct sequence analysis on an ABI 310 sequencer.

For sequence analysis of relevant regions of the TPMT gene (Seki, J Hum Genet 45 (2000), 299-302; Szumianski, DNA Cell Biol 15 (1996), 17-30), PCR amplifications of the relevant regions of the gene were carried out (primers see table 1), following purification of the PCR products and sequencing with established methods (ABI Big Dye terminator cycle sequencing). Since the individual genetic makeup is represented by two copies of any gene (diploidy), great care has to be taken in the evaluation of the sequences not to unambiguously identify homozygous, but also heterozygous sequence variation. Therefore in all cases forward and reverse sequencing was performed and to confirm new allelic variants genomic DNA was taken for a second PCR and sequencing procedure was repeated.

The sequences were subjected to a computer analysis program (ABI DNA sequencing analysis Software 3.3) and inspected manually for the occurrence of DNA sequences deviating from published TPMT sequences that were considered to represent the wild type sequences in this work. Using this approach six new sequence variations were discovered and experimentally confirmed as shown in table 2.

Six novel SNPs could be identified that change the protein sequence as shown in table 3.

The positions of the novel TPMT SNPs, including the exact sequence context are listed in table 2. The deviative base in the sequence is underlined and in bold style.

As shown in FIG. 1, the eight individuals described in this work containing the novel mutations leading to reduced or low TPMT activity. TPMT enzyme activity was measured by high-performance liquid chromatography method as described previously by Kroeplin et al. (Kroplin, Eur J Clin Pharmacol 55 (1999), 285-91; Kroplin, Eur J Clin Pharmacol 54 (1998), 265-71). This method was validated and compared to the standard radiochemical assay of TPMT activity as described by Weinshilboum et al. (Weinshilboum, Am J Hum Genet 32 (1980), 651-62). Very low enzyme levels (<2 nmol 6-methylthioguanine×g⁻¹Hb×h⁻¹) are indicative for TPMT deficiency, the cut-off value for intermediate to high TPMT activity was set at 24 nmol 6-methylthioguanine×g⁻¹Hb×h⁻¹ based on analysis data from healthy blood donors (n=1200). (<2 deficient, 2-24 intermediate, >24 wildtype). The cut-off value of 24 corresponds to 13.7 nmol 6-methylmercaptopurine×ml⁻¹ RBC×h⁻¹ obtained by the classical radiochemical assay (<4.7 deficient, 5.2-13.7 intermediate, >13.9 wildtype). In the present study of healthy blood donors, one child with ALL and in one patient with thiopurine therapy due to inflammatory bowel disease the reduced and very low TPMT activity in the described eight individuals can not be explained by the presence of all known mutant alleles, associated with TPMT deficiency. Thus reduced or low TPMT activity in these individuals can be explained by the presence of the above described novel SNPs.

### EXAMPLE 2

### Correlation of New SNPs with Reduced TMPT Activity

In the past 30 years it has been established that genetic polymorphisms of drug metabolizing enzymes contribute in a major way to dose-dependent drug toxicity as well as drug response for numerous agents (e.g. propafenone, antidepressants, warfarin, omeprazole). In the case of TPMT which was one of the first pharmacogenetically investigated enzymes, polymorphisms may be routinely considered for the drug treatment with thiopurines. As a consequence, phenotyping of TPMT has become standard practice in major cancer treatment centers (Mayo Clinic, Rochester, Minn. and the St. Jude's Children Research Hospital, Memphis, Tenn.). However, several authors mentioned the possibility of a misclassification of TPMT phenotype in patients who had received RBC transfusion from a homozygous wildtype individual by using measurement of TPMT activity only (Schwab, Gastroenterology 121 (2001), 498-499; Yates, Ann Intern Med 126 (1997), 608-614; Krynetski, Pharmacology 61 (2000), 136-146). This is a rather likely scenario since approximately 90% of Caucasians as well as African-Americans have TPMT wildtype and subsequently show high enzyme activity. Thus, for rapid and unequivocal determination of the genuine TPMT phenotype of patients receiving blood transfusion, genotyping of TPMT appears to be the only reliable method. It must be assured that individuals homozygous for TPMT are correctly detected by genotyping and thereby predicting a patient's phenotype to almost 100%. To date, phenotype-genotype correlation studies suggest that genotyping for TPMT is predictive in only up to 95% for low enzyme activity. Ten mutant alleles are described to be associated with intermediate or low activity of TPMT. The six above described nucleotide substitutions in the TPMT gene lead to low TPMT activity as shown in FIG. 1. By Including the six nucleotide substitutions described in table 2, phenotype-genotype correlations up to >99% may be achieved.

1. A polynucleotide comprising a polynucleotide selected from the group consisting of:
(a) a polynucleotide having the nucleic acid sequence of SEQ ID NO: 7 to 18;
(b) a polynucleotide encoding a polypeptide having the amino acid sequence of SEQ ID NO: 19 TO 24;
(c) a polynucleotide capable of hybridizing to a TPMT gene, wherein said polynucleotide has a substitution at a position corresponding to position 488 of the TPMT gene (GenBank Accession No: AF019364.1); or at a position corresponding to position 516 of the TPMT gene (GenBank Accession No: AF019367.1); or at a position corresponding to position 391 of the TPMT gene (GenBank Accession No: AF019365.1); or at a position corresponding to position 463 of the TPMT gene (GenBank Accession No: AF019366.1), or at a position corresponding to position 1236 of the TPMT gene (GenBank Accession No: AF019367.1); or at a position corresponding to position 679 of the TPMT gene (GenBank Accession No: AF019369.1);
(d) a polynucleotide capable of hybridizing to a TPMT gene, wherein said polynucleotide has a G at a position corresponding to position 488 of the TPMT gene (GenBank Accession No: AF019364.1) or an A at a position corresponding to position 391 of the TPMT gene (GenBank Accession No: AF019365.1) or an A at a position corresponding to position 516 or 1236 of the TPMT gene (GenBank Accession No: AF019367.1) or a C at a position corresponding to position 463 of the TPMT gene (GenBank Accession No: AF019366.1) or a G at a position corresponding to position 679 of the TPMT gene (GenBank Accession No: AF019369.1);
(e) a polynucleotide encoding an TPMT polypeptide or fragment thereof, wherein said polypeptide comprises an amino acid substitution at position 42, 71, 119, 132, 163 or 238 of the TPMT polypeptide (GenBank Accession No: AAC51865.1);
(f) a polynucleotide encoding an TPMT polypeptide or fragment thereof, wherein said polypeptide comprises an amino acid substitution of Q to E at position 42 of the TPMT polypeptide (GenBank Accession No: AAC51865.1) or G to R at position 71, or K to T at position 119 or C to Y at position 132 or R to H at position 163 or K to E at position 238 of the TPMT polypeptide (GenBank Accession No: AAC51865.1).

2. A polynucleotide of claim 1, wherein said polynucleotide is associated with a TPMT associated disease.

3. A polynucleotide of claim 1 which is DNA or RNA.

4-5. (canceled)

6. A vector comprising a polynucleotide of claim 1.

7. The vector of claim 6, wherein the polynucleotide is operatively linked to expression control sequences allowing expression in prokaryotic or eukaryotic cells or isolated fractions thereof.

8. A host cell genetically engineered with the polynucleotide of claim 1.

9. A method for producing a molecular variant TPMT polypeptide or fragment thereof comprising
(a) culturing the host cell of claim 8; and
(b) recovering said protein or fragment from the culture.

10. A method for producing cells capable of expressing a molecular variant TPMT polypeptide comprising genetically engineering cells with the polynucleotide of claim 1.

11. A polypeptide or fragment thereof encoded by the polynucleotide of claim 1.

12. An antibody which binds specifically to the polypeptide of claim 11.

13-18. (canceled)

19. An in vitro method for identifying a single nucleotide polymorphism said method comprising the steps of:
(a) isolating a polynucleotide of claim 1 from a plurality of subgroups of individuals, wherein one subgroup has no prevalence for a TPMT associated disease and at least one or more further subgroups do have prevalence for a TPMT associated disease; and
(b) identifying a single nucleotide polymorphism by comparing the nucleic acid sequence of said polynucleotide or said gene of said one subgroup having no prevalence for a TPMT associated disease with said at least one or more further subgroups having a prevalence for a TPMT associated disease.

20-28. (canceled)

29. A method of diagnosing a TPMT associated disease or susceptibility therefor comprising determining the presence of a polynucleotide of claim 1 in a sample from a subject.

30. The method of claim 29 further comprising determining the presence of a polypeptide of claim 11 or an antibody which binds specifically to the polypeptide of claim 11.

31. A method of diagnosing a TPMT associated disease or susceptibility therefor comprising determining the presence of a polypeptide of claim 11 or an antibody which binds specifically to the polypeptide of claim 11 in a sample from a subject.

32. The method of claim 29, wherein said disorder is thiopurine-induced toxicity, myelosuppression (pancytopenia) and gastrointestinal disturbances, leucopenia, thrombocytopenia, anemia.

33. The method of claim 29 comprising PCR based techniques, RFLP-based techniques, DNA sequencing-based techniques, hybridization techniques, Single strand conformational polymorphism (SSCP), denaturating gradient gel electrophoresis (DGGE), mismatch cleavage detection, heteroduplex analysis, techniques based on mass spectroscopy, HPLC-based techniques, primer extension-based techniques, and 5′-nuclease assay-based techniques.

34-39. (canceled)

40. A polynucleotide of claim 2, wherein said TPMT associated disease is thiopurine-induced toxicity.

41. The polynucleotide of claim 40, wherein said thiopurine-induced toxicity is myelosupression.

42. The polynucleotide of claim 41, wherein myelosupression is leucopenia, thrombocytopenia or anemia.

43. (canceled)

44. A vector comprising the gene of claim 4.
